(19) United States
(12) Patent Application Publication
(10) Pub. No.: US 2011/0270020 A1
(43) Pub. Date: Nov. 3, 2011

(54) Linear tension internal organ supports and method for using the same

(76) Inventors: Brian B. Quebbemann, Newport Beach, CA (US); Clark B. Foster, Mission Viejo, CA (US)

(21) Appl. No.: 12/799,694

(22) Filed: Apr. 30, 2010

(51) Int. Cl. A61F 2/00

(52) U.S. Cl. 600/37

(57) ABSTRACT

Compact and efficient linear tension internal organ supports and methods of maneuvering the organ supports so as to manipulate (i.e., lift, suspend or displace) an internal organ or soft tissue within the general operating field within the abdominal cavity of a patient undergoing surgery. In certain preferred embodiments, an internal organ support is attached directly to the organ or tissue to be manipulated or to the abdominal wall within the abdominal cavity. In other preferred embodiments, a displacement force is applied to an internal organ support from a location outside the patient's abdomen by way of linear tension lines attached to the organ support and removed from the abdominal cavity through a small exit incision made through the abdominal wall. By virtue of the internal organ supports and methods herein disclosed, only a single, small entry incision need be made so that only a small scar or no visible scar at all will remain on the patient's abdominal skin after the internal organ supports have been removed from the abdominal cavity following the surgery.

### BACKGROUND OF THE INVENTION

1. Field of the Invention

This invention relates to internal organ supports and methods for manipulating the organ supports within the abdominal cavity of a patient undergoing surgery in order to lift and move an organ or soft tissue within the general operating field. By virtue of the internal organ supports herein disclosed, only a single, small entry incision need be made so that only a small scar or no visible scar at all will remain in the patient's abdominal skin after the internal organ supports have been removed from the abdominal cavity at the conclusion of the surgery.

2. Background Art

Single incision procedures are known to surgeons who operate within the abdominal cavity of a patient. However, the single incision that is made through the abdominal wall is typically very long so as to be able to accommodate all of the necessary fixed access instruments required for surgery. By way of example, generally large and cumbersome laparoscopic instruments, one or more of which having a curved configuration, are often inserted through an incision in the patient's abdomen. Because the surgeon must first move an organ in order to gain access to the actual operating site, a number of different instruments are usually inserted at the same time. In some cases, more than a single incision may be required to accommodate all of the tools necessary for surgery. Consequently, a correspondingly long and often unsightly scar may remain in the patient's abdomen after the surgery has been completed. What is more, a longer incision may introduce tissue trauma and increase risks to the patient. As a result of the potential for risk and scarring, some patients may elect to forego surgery altogether.

Accordingly, it would be desirable to be able to minimize the number of instruments required within the patient's abdomen during surgery so that the size of the entrance incision can be correspondingly reduced to minimize tissue trauma, risk to the patient, and scarring of the abdomen following surgery.

### SUMMARY OF THE INVENTION

Compact internal organ supports including tissue engaging and manipulation devices are disclosed that are adapted to be positioned by a surgeon within the abdominal cavity of a patient undergoing surgery so as to apply a perpendicular lifting force or a linear retraction for manipulating an organ or soft tissue of the patient within the operative field to give the surgeon better visibility and easier access thereto. In this manner, an organ or tissue can be manipulated relative to the operating field to allow surgical procedures to be performed by using only a minimal number of conventional (e.g., laparoscopic) instruments which pass through a single access incision. Because of their compact nature, the internal organ supports herein disclosed can be advantageously inserted through a single small incision, puncture of a natural orifice.

A first internal organ support to be temporarily implanted within the abdominal cavity of a patient undergoing surgery includes an intra-abdominal tissue-grasping element having a pair of articulating jaws that are moved to a closed position into grasping engagement with an organ or tissue to be manipulated. One end of a linear tension line is tied to the tissue-grasping element. The opposite end of the tension line exits the abdominal cavity through a small incision made in the abdominal wall so as to be held outside the patient's abdomen by means of an external fixation clamping instrument. The tension line is kept in a state of constant linear tension between the external fixation clamping instrument and the intra-abdominal tissue-grasping element so as to hold the organ or tissue above the operating field.

A second internal organ support to be temporarily implanted within the patient's abdominal cavity includes a tension line that is surrounded by a soft flexible transabdominal tube or sleeve positioned underneath the organ or tissue to be manipulated. The opposite ends of the tension line exit the abdominal cavity through small incisions made in the abdominal wall to be held outside the patient's abdomen by respective external fixation clamping instruments. The tension line is maintained in a state of linear tension between the external fixation clamping instruments to cause the transabdominal tube to provide lateral support and exert a perpendicular lifting force by which to hold the organ or tissue above the operating field.

A third internal organ support to be temporarily implanted within the patient's abdominal cavity includes a linear tension line that is surrounded by a soft, flexible transabdominal tube or sleeve to be positioned underneath the organ or tissue to be manipulated. The opposite ends of the tension line are tied to respective intra-abdominal tissue-grasping elements, each of which having a pair of articulating jaws that are moved to a closed position into grasping engagement with the abdominal wall. The tension line is held in a state of linear tension between the intra-abdominal tissue-grasping element to cause the transabdominal tube to provide lateral support and exert a perpendicular lifting force by which to hold the organ or tissue above the operating field.

A fourth internal organ support to be temporarily implanted within the patient's abdominal cavity includes a flexible cradle body positioned underneath the organ or tissue to be lifted. A plurality of support arms extend from the cradle body, and respective linear tension lines are attached at first ends thereof to the support arms. Opposite ends of some of the linear tension lines are tied to intra-abdominal tissue-grasping elements having a pair of articulating jaws that are moved to a closed position into grasping engagement with the abdominal wall. Opposite ends of other ones of the tension lines exit the abdominal cavity through small incisions made in the abdominal wall so as to be held outside the patient's abdomen by respective external fixation clamping instruments. The tension lines attached to the support arms of the cradle body are maintained in a state of tension by the intra-abdominal tissue-grasping elements and the external fixation clamping instruments to cause the cradle body to provide lateral support and a lifting force by which to hold the organ or tissue above the operating field.

### BRIEF DESCRIPTION OF THE DRAWINGS

FIG. 1 shows an internal organ or tissue support temporarily implanted within the abdominal cavity of a patient undergoing surgery according to a first preferred embodiment of this invention for engaging and manipulating an organ or tissue within an operating field;

FIG. 2 shows an internal organ or tissue support temporarily implanted within the abdominal cavity of a patient undergoing surgery according to a second preferred embodiment of this invention for supporting and manipulating an organ or tissue within an operating field;

FIG. 3 shows an internal organ or tissue support temporarily implanted within the abdominal cavity of a patient undergoing surgery according to a third preferred embodiment of this invention for supporting and manipulating an organ or tissue within an operating field;

FIG. 4 shows an internal organ or tissue support temporarily implanted within the abdominal cavity of a patient undergoing surgery according to a fourth preferred embodiment of this invention for supporting and manipulating an organ or tissue within an operating field;

FIG. 5 shows an intra-abdominal tissue-grasping element from the internal organ and tissue supports of FIGS. 1, 3 and 4 to be moved into grasping engagement with the organ or tissue or the abdominal wall within the patient's abdominal cavity; and

FIG. 6 is a cross-section of the intra-abdominal tissue-grasping element taken along lines 6-6 of FIG. 5.

### DESCRIPTION OF THE PREFERRED EMBODIMENT

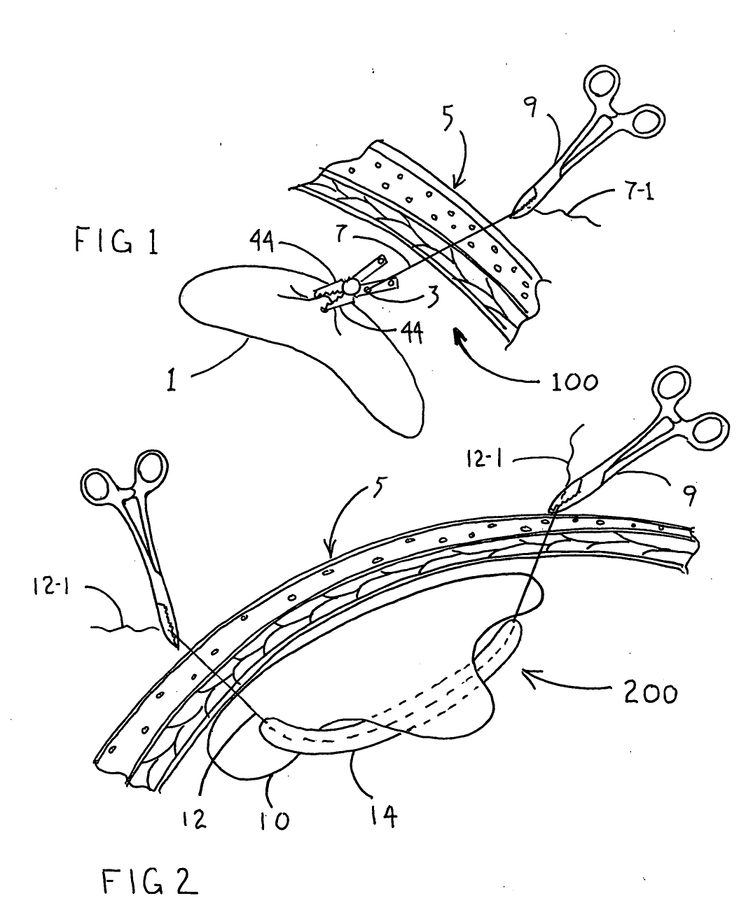

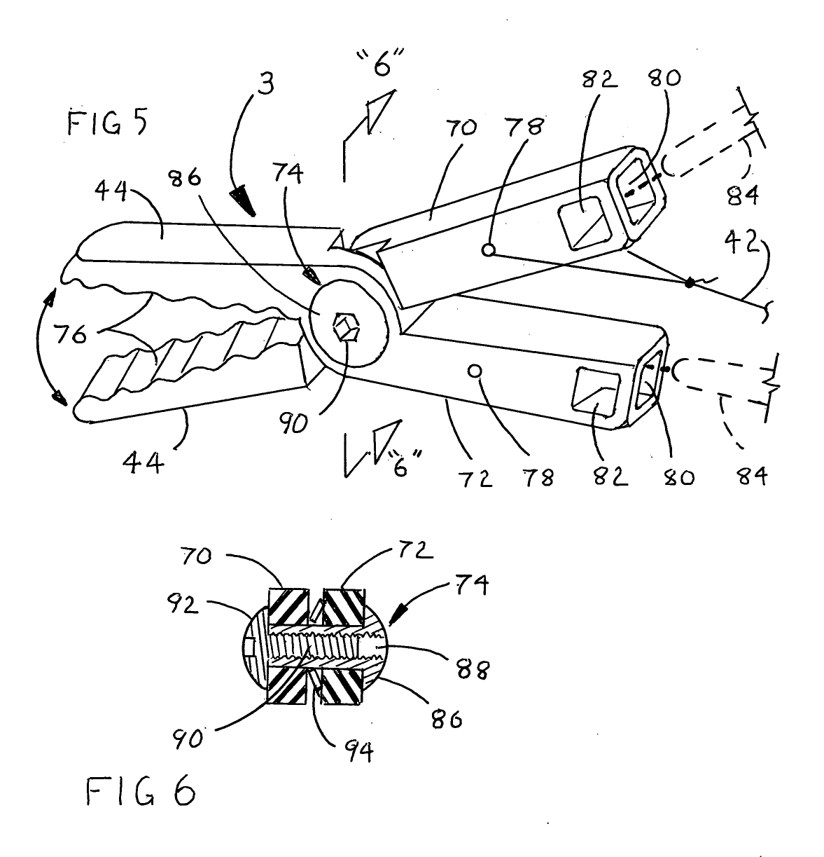

Referring initially to FIG. 1 of the drawings, a method is described and a compact and efficient linear tension internal organ support 100 is shown temporarily implanted within the abdominal cavity of a patient according to a first preferred embodiment of this invention to enable a surgeon to lift and/or move an internal organ 1 or other soft tissue above and within an operating field during a surgical procedure. The internal organ support 100 includes an intra-abdominal tissue grasping element 3 that is inserted into the abdominal cavity. The intra-abdominal tissue grasping element 3 will be described in greater detail hereinafter when referring to FIGS. 5 and 6. A linear tension line 7 is tied at one end thereof to the tissue grasping element 3. By way of example, the tension line 7 may be an internal suture material that is sold commercially as Ethibond manufactured by Johnson & Johnson. The tissue grasping element 3 has a pair of articulating jaws 44 that are moved by the surgeon to a closed position at which to grasp the organ 1 to be lifted or moved in the general operating field. The opposite free end of the tension line 7 exits the abdominal cavity (e.g., by means of a surgical grade needle, grasper or the like) through a small incision that is made in the abdominal wall 5 or through the incision that is made to implant the internal organ support 100.

The free end of the tension line 7 is engaged and held outside the patient's abdomen by a commercially-available external fixation (e.g., clamping) instrument 9. The clamping instrument 9 maintains a pulling force to the external free end of tension line 7, whereby the tension line will be held in a state of constant linear tension. Accordingly, the organ 1 or tissue that is engaged by the intra-abdominal tissue grasping element 3 can be selectively manipulated (i.e., lifted, suspended or displaced) by the surgeon above and around the general operating field depending upon a corresponding manipulation of the external clamping instrument 9 and the resulting tension applied to the tension line 7.

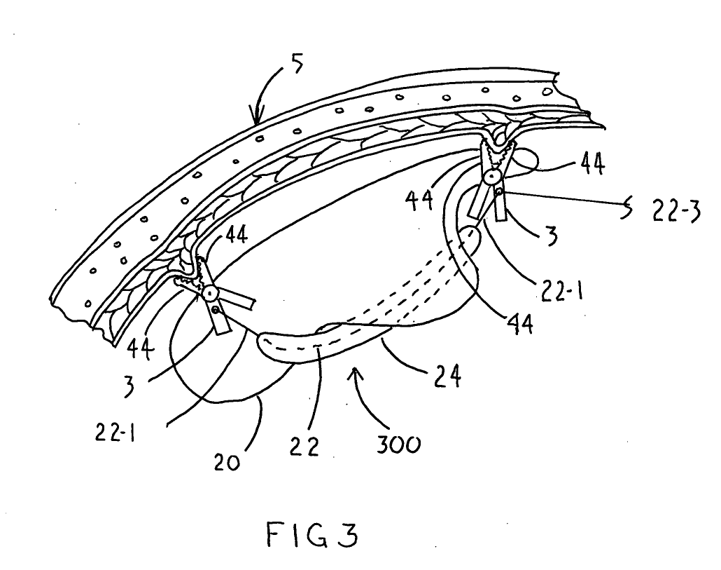

Turning to FIG. 2 of the drawings, a method is described and a compact and efficient linear tension internal organ support 200 is shown temporarily implanted within the abdominal cavity of a patient according to a second preferred embodiment of this invention to enable a surgeon to lift and/or move an internal organ 10 or soft tissue above and within the general operating field during a surgical procedure. The internal organ support 200 includes a linear tension line 12 like that shown and described while referring to FIG. 1. In this case, however, the tension line 12 is surrounded by a soft, flexible (e.g., elastomeric) transabdominal tube or sleeve 14. Rather than tie the tension line 12 to an intra-abdominal tissue grasping element like that designated 3 in FIG. 1, each of the opposite free ends 12-1 of the tension line 12 exit the abdominal cavity (e.g., by means of a surgical grade needle, grasper or the like) through a small incision that is made in the abdominal wall 5 or through the incision that is made to implant the internal organ support 200.

The external free ends 12-1 of the tension line 12 are grasped outside the patient's abdomen by suitable external fixation clamping instruments 9 like that shown in FIG. 1. The clamping instruments 9 are manipulated by the surgeon to hold the tension line 12 in a state of linear tension. One or both of the clamping instruments 9 are also manipulated so that the flexible transabdominal tube 14 surrounding the tension line 12 will run underneath the organ 10 to be lifted or moved. In the case of the organ support 200 of FIG. 2, the flexible transabdominal tube 14 that is located within the abdominal cavity functions as a sling to provide lateral support for and apply a perpendicular lifting force to the organ 10 or tissue. Accordingly, the organ or tissue that is supported by the flexible tube 14 of organ support 200 can be selectively manipulated (i.e., lifted, suspended or displaced) by the surgeon above and around the general operating field depending upon a corresponding manipulation of the external clamping instruments 9 and the resulting tension that is applied to the tension line 12 and the flexible tube 14 carried thereby.

Moving now to FIG. 3 of the drawings, a method is described and a compact and efficient linear tension internal organ support 300 is shown temporarily implanted within the abdominal cavity of a patient according to a third preferred embodiment of this invention to enable a surgeon to lift and/or move an internal organ 20 or soft tissue above and within the general operating field during a surgical procedure. Like the organ support 200 of FIG. 2, the organ support 300 of FIG. 3 includes a linear tension line 22 that is surrounded by a soft, flexible transabdominal tube or sleeve 24. In the case of the organ support 300, the opposite ends 22-1 of the tension line 22 are tied to respective intra-abdominal tissue grasping elements 3, like that shown in FIG. 1.

Each of the tissue grasping elements 3 has a pair of articulating jaws 44 that are moved by the surgeon to a closed position at which to grasp the abdominal wall 5. As with the organ support 200 of FIG. 2, the flexible transabdominal tube 24 of the organ support 300 of FIG. 3 is positioned within the abdominal cavity to function as a sling and provide lateral support for and apply a perpendicular lifting force to the organ 20 or tissue in response to the tension line 22 being held in a state of linear tension between the intra-abdominal tissue grasping elements 3. Thus, the organ or tissue that is supported by the flexible tube 24 of the internal organ support 300 can be selectively manipulated (i.e., lifted, suspended or displaced) by the surgeon above and around the general operating field depending upon the corresponding manipulation of the tissue grasping elements 3 and the particular location at which the grasping elements are attached to the patient's abdominal wall 5.

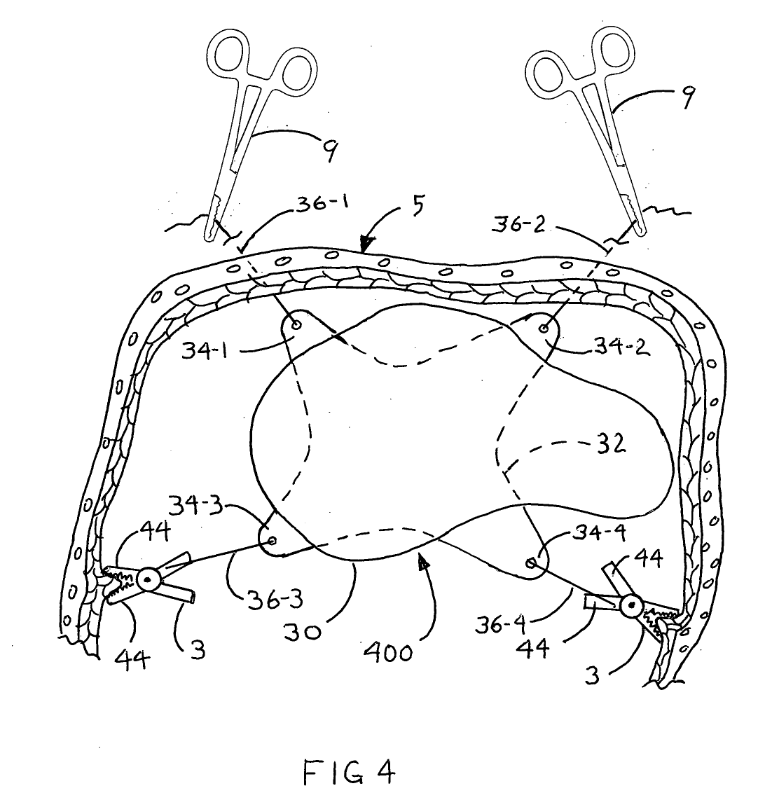

Referring now to FIG. 4 of the drawings, a method is described and a compact and efficient linear tension internal organ support 400 is shown temporarily implanted within the abdominal cavity of a patient according to a fourth preferred embodiment of the present invention to enable a surgeon to lift and/or move an internal organ 30 or soft tissue above and within the general operating field during a surgical procedure. The internal organ support 400 includes a relatively wide cradle body 32 that is preferably manufactured from a cloth (e.g., Dacron mesh or nylon web) material. In the configuration of FIG. 4, the cradle body 32 has four stabilizing arms 32-1 . . . 32-4 projecting outwardly therefrom. A linear tension line 36-1 . . . 36-4 is attached (e.g., tied) at respective first ends thereof to each of the stabilizing arms 34-1 . . . 34-4 of cradle body 32.

The opposite end of each of a first pair of tension lines 36-1 and 36-2 exits the abdominal cavity (e.g., by means of a surgical grade needle, grasper or the like) through a small incision that is made in the abdominal wall 5 or through the incision that is made to implant the internal organ support 400. The external free ends of the tension lines 36-1 and 36-2 are grasped by external clamping instruments 9 like those shown in FIGS. 1 and 2. The clamping instruments 9 are manipulated by the surgeon outside the patient's abdomen to hold the tension lines 36-1 and 36-2 as well as the stabilizing arms 34-1 and 34-2 of the cradle body 32 to which the tension lines are tied in a state of linear tension. One or both of the clamping instruments 9 can also be manipulated so that the cradle body 32 of the internal organ support 400 will be located under the organ 30 to be lifted or moved.

The opposite end of each of the second pair of tension lines 36-3 and 36-4 is tied to an intra-abdominal tissue grasping element 3 like that shown in FIGS. 1 and 3. As previously described, each of the tissue grasping elements 3 has a pair of articulating jaws 44 that are moved by the surgeon to a closed position at which to grasp the abdominal wall 5. The internal organ support 400 is particularly useful to embrace a large organ 30 or tissue so as to provide lateral support for and apply a lifting force to the organ or tissue in response to the tension lines 36-1 . . . 36-4 and the respective stabilizing arms 34-1 . . . 34-4 of cradle body 32 being held in a state of linear tension by the external clamping instruments 9 and the intra-abdominal grasping elements 3.

Thus, the organ or tissue that is carried and supported by the cradle body 32 of organ support 400 can be selectively manipulated (i.e., lifted, suspended or displaced) by the surgeon above and around the general operating field depending upon a corresponding manipulation of the external clamping instruments 9 and the particular location at which the intra-abdominal tissue grasping elements 3 are attached to the patient's abdominal wall 5.

Each of the linear organ support 100, 200, 300 and 400 shown in FIGS. 1-4 may be pushed through a single, relatively small incision that is made by the surgeon in order to be able to engage and hold the organ or tissue off the operating field in order to give the surgeon better access thereto. In particular, the organ supports can be inserted through a puncture, an incision, a central abdominal access port at the umbilicus, or a natural orifice such as the nasal passage. By virtue of its compact nature, a surgeon can use existing tools (e.g., laparoscopic instruments) to implant the organ supports 100, 200, 300 and 400 within the patient's abdominal cavity. In each of the examples described above when referring to FIGS. 1-4, the entry point can be remote from the implanted organ support 100, 200, 300 and 400. Following the surgical procedure, the same tools used for implantation may also be used to grasp, manipulate and remove the organ supports from the abdominal cavity by way of the original entry point. Depending upon the organ or tissue to be manipulated, two or more linear tension organ supports 100, 200, 300 and 400 may be required to evenly distribute the lifting force along the organ. In fact, different ones of the organ supports shown in FIGS. 1-4 may be used in combination with one another within the patient's abdominal cavity during the same surgical procedure.

Details of one of the intra-abdominal tissue grasping clamps 3 shown in FIGS. 1, 3 and 4 for attaching the organ support directly to the organ or to the abdominal wall 5 within the patient's abdominal cavity are now disclosed while referring to FIGS. 5 and 6 of the drawings. The tissue clamp 3 includes a pair of lever arms 70 and 72 that are pivotally connected to one another at a friction pivot axis assembly 74. First ends of the lever arms 70 and 72 are grips to be manipulated by the surgeon. The opposite ends of the lever arms 70 and 72 form the articulating jaws 44 that are adapted to be rotated towards one another around the pivot axis assembly 74 between an open position and a closed position in order to engage and grasp the organ 1 (as shown in FIG. 1) or the abdominal wall 5 (as shown in FIGS. 3 and 4) and thereby create a lifting force by way of a linear tension line in the manner described above. Each of the jaws 44 of lever arms 70 and 72 preferably includes serrations 76 to enhance the ability of the tissue grasping clamp 3 to remain attached to the organ 1 or abdominal wall 5.

Each of the grips at the first ends of the lever arms 70 and 72 of the intra-abdominal tissue grasping clamp 3 includes a hole 78 extending laterally therethrough. One end of a tension line of the organ supports 100, 300 and 400 in FIGS. 1, 3 and 4 is tied to or routed through the clamp 3 at the hole 78 running through one of the arms 70 or 72. Each of the first ends of the lever arms 70 and 72 also includes a pocket 80 and a window 82. The pockets 80 extend longitudinally within the lever arms 70 and 72, and the windows 82 run laterally therethrough, such that the pockets 80 and windows 82 are arranged in perpendicular alignment. The pockets 80 and windows 82 are sized to receive the tips (shown in phantom lines designated 84 in FIG. 5) of surgical instruments to be manipulated by the surgeon. That is to say, a force applied by the surgeon to the instrument tips 84 located within the pockets 80 (as shown) or the windows 82 of lever arms 70 and 72 will cause the articulating jaws 44 to rotate at the friction pivot axis assembly 74 towards or away from one another between the open and closed positions depending upon whether the clamp 3 is being attached to or removed from the organ or the abdominal wall within the patient's abdominal cavity.

In the case where the articulating jaws 44 are rotated towards one another, the friction pivot axis assembly 74 holds the jaws 44 in the closed position so as to reliably grasp the organ or abdominal wall. FIG. 6 of the drawings shows the friction pivot axis assembly 74 including a headed axle 86 having a threaded bore 88 that is received through the lever arms 70 and 72 to establish a pivot axis around which the arms rotate. The threaded body 90 of a screw 92 is rotated axially through and into mating engagement with the threaded bore 88 of axle 86. A spring (e. g. bevel or wave body type) washer 94 is disposed between the lever arm 70 and 72 in surrounding engagement with the axle 86. The spring characteristic of the washer 94 exerts a frictional pushing force against the lever arms 70 and 72 by which the articulating jaws 44 of the intra-abdominal tissue grasping clamp 3 can be reliably attached to the organ or the abdominal wall until such time as when the surgery is completed and the clamp 3 is detached and removed from the abdominal cavity as was earlier described.

By virtue of the internal organ supports 100, 200, 300 and 400 herein disclosed and the methods for implanting and manipulating the supports, only minimal access to the abdominal cavity is necessary, such that a single, relatively small entry wound need be made which will leave an imperceptible scar or no visible scar at all at an area of the patient's body which can be remote from the entry site. That is, the need to introduce multiple and cumbersome rigid graspers, retractors, and similar fixed access surgical instruments through separate and/or long incisions through the abdomen is eliminated. Organ supports will now be available which can be manipulated by the primary surgeon internally or by a robotic device that is located externally or remotely from the patient. By way of example only, the disclosed organ supports will be particularly advantageous to gallbladder surgeries, bariatric surgeries, gynecological surgeries, and other abdominal surgeries which are intended to be minimally invasive. The organ supports of this invention are capable of providing low-pressure support for an organ or other soft tissue by means of linear tension, whereby the organ or tissue will experience less trauma to help speed the patient's recovery time.

1. A method for moving an internal organ or tissue within an operating field located within the abdominal cavity of a patient undergoing surgery, said method comprising the steps of implanting an intra-abdominal organ-manipulating element within the abdominal cavity, positioning said organ-manipulating element within the abdominal cavity so as to engage the internal organ or tissue to be moved, applying a tension force to said organ-manipulating element to cause the organ to be moved within the operating field, and withdrawing said organ-manipulating element from the abdominal cavity at the conclusion of the surgery.

2. The method recited in claim 1, comprising the additional step of generating the tension force applied to said intra-abdominal organ-manipulating element implanted within the patient's abdominal cavity from a location outside the patient's abdomen.

3. The method recited in claim 2, comprising the additional step of applying the tension force to said intra-abdominal organ-manipulating element by way of at least one tension line coupled at a first end thereof to said organ-manipulating element.

4. The method recited in claim 3, comprising the additional step of removing the opposite end of said one tension line coupled to said intra-abdominal organ-manipulating element to said location outside the patient's abdomen via an exit path made in the abdominal wall.

5. The method recited in claim 4, including the additional step of attaching an external clamp to the opposite end of said one tension line at said location outside the patient's abdomen by which to hold said tension line in a state of linear tension and thereby cause said tension force to be applied to said intra-abdominal organ-manipulating element implanted within the patient's abdominal cavity.

6. The method recited in claim 4, wherein said intra-abdominal organ-manipulating element implanted within the patient's abdominal cavity is a clamp having a pair of articulating jaws moving from an open position to a closed position at which to grasp the internal organ or tissue to be moved within the operating field.

7. The method recited in claim 6, wherein the first end of said one tension line is tied to said intra-abdominal organ-manipulating clamp implanted within the patient's abdominal cavity.

8. The method recited in claim 6, wherein said intra-abdominal organ-manipulating clamp has a pair of lever arms pivotally coupled to each other and communicating with respective ones of said pair of articulating jaws, said method comprising the additional step of applying a force to each of said pair of lever arms to cause said pair of jaws to rotate relative to one another from the open position to the closed position.

9. The method recited in claim 6, wherein each of said pair of lever arms of said intra-abdominal organ-manipulating clamp has a force-receiving pocket formed therein, said method comprising the additional step of applying said force to each of said lever arms at said force-receiving pocket thereof.

10. The method recited in claim 4, wherein said intra-abdominal organ-manipulating element implanted within the patient's abdominal cavity includes a flexible tube, said method comprising the additional steps of positioning said flexible tube within the patient's abdominal cavity to lie underneath and support the internal organ or tissue to be moved, and applying a pulling force to the opposite end of said one tension line at said location outside the patient's abdomen to hold said tension line in a state of linear tension and thereby cause said flexible tube to apply a lifting force to the patient's organ or tissue.

11. The method recited in claim 10, wherein said flexible tube surrounds said one tension line.

12. A method for moving an internal organ or tissue within an operating field located within the abdominal cavity of a patient undergoing surgery, said method comprising the steps of implanting an intra-abdominal organ-manipulating element within the abdominal cavity, positioning said intra-abdominal organ-manipulating element within the abdominal cavity so as to lie underneath and support the internal organ or tissue to be moved, attaching said organ-manipulating element to the abdominal wall within the patient's abdominal cavity so that the organ or tissue is suspended above the operating field, and withdrawing said organ-manipulating element from the abdominal cavity at the conclusion of the surgery.

13. The method recited in claim 12, wherein said intra-abdominal organ-manipulating element is attached to the abdominal wall within the patient's abdominal cavity by way of at least one tension line coupled at a first end thereof to said organ-manipulating element.

14. The method recited in claim 13, comprising the additional step of attaching said intra-abdominal organ-manipulating element to the abdominal wall within the patient's abdominal cavity by means of a clamp having a pair of articulating jaws moving from an open position to a closed position at which to grasp the abdominal wall, the opposite end of said one tension line being attached to said clamp.

15. The method recited in claim 14, wherein said intra-abdominal organ-manipulating clamp has a pair of lever arms pivotally coupled to each other and communicating with respective ones of said pair of articulating jaws, said method comprising the additional step of applying a force to each of said pair of lever arms to cause said pair of jaws to rotate relative to one another from the open position to the closed position.

16. The method recited in claim 14, wherein each of said pair of lever arms of said intra-abdominal organ-manipulating clamp has a force-receiving pocket formed therein, said method comprising the additional step of applying said force to each of said lever arms at said force-receiving pocket thereof.

17. The method recited in claim 12, wherein said intra-abdominal organ-manipulating element implanted within the patient's abdominal cavity is a flexible tube.

18. The method recited in claim 17, wherein said flexible tube surrounds said one tension line.

19. A method for moving an internal organ or tissue within an operating field located within the abdominal cavity of a patient undergoing surgery, said method comprising the steps of implanting an intra-abdominal organ-manipulating element within the abdominal cavity so as to engage the patient's organ or tissue, attaching a first portion of said organ-manipulating element to the abdominal wall within the patient's abdominal cavity, applying a tension force to a second portion of said organ-manipulating element from a location outside the patient's abdomen so that the organ is suspended above the operating field, and withdrawing the organ-manipulating element from the abdominal cavity at the conclusion of the surgery.

20. The method recited in claim 19, comprising the additional step of attaching the first portion of said intra-abdominal organ-manipulating element to the abdominal wall within the patient's abdominal cavity by means of a clamp having a pair of articulating jaws moving from an open position to a closed position at which to grasp the abdominal wall.

21. The method recited in claim 19, comprising the additional step of applying said tension force to the second portion of said intra-abdominal organ-manipulating element at said location outside the patient's abdomen by way of at least one tension line attached at one end thereof to the second portion of said organ-manipulating element.

22. The method recited in claim 21, comprising the additional step of removing the opposite end of said one tension line attached to the second portion of said intra-abdominal organ-manipulating element to said location outside the patient's abdomen via an exit path through the abdominal wall so as to receive said tension force.
